(19) United States
(12) Patent Application Publication
(10) Pub. No.: US 2021/0322798 A1
(43) Pub. Date: Oct. 21, 2021

(54) Cartridge for Respiration Device

(71) Applicant: Xicore Inc., Mississauga (CA)

(72) Inventor: Ray ELEID, Mississauga (CA)

(21) Appl. No.: 17/236,867

(22) Filed: Apr. 21, 2021

Related U.S. Application Data
(60) Provisional application No. 63/013,434, filed on Apr. 21, 2020.

(51) Int. Cl. A62B 7/10; A62B 19/00; A62B 23/02; A62B 18/02; A61L 9/20

(52) U.S. Cl. CPC A62B 7/10; A62B 19/00; A62B 23/02; A61L 2209/14; A61L 9/20; A61L 2209/12; A62B 18/025

(57) ABSTRACT

A respiration device is provided configured to kill infectious virus or other air borne contagion before it enters the user's airways. The respiration device comprises a wearable mask portion, an evacuation filter in fluid connection to the wearable mask portion; and at least one UV (ultraviolet) cartridge in fluid connection to the wearable mask portion. The UV cartridge comprises a housing having an inlet at a first end and an outlet at a second end opposite the first end; a series of UV bulbs located within the housing; and a powering means. Ambient air enters the respirator device through the inlet and is subject to UV radiation emitted from the series of UV bulbs such that the ambient air becomes sterilized air. Sterilized air exits the UV cartridge and enters the wearable mask portion via the outlet. A user is able to inhale the sterilized air entering the mask portion and exhale the used air through the evacuation filter.

### CROSS-REFERENCE TO RELATED APPLICATIONS

The present application claims priority from U.S. Provisional Application No. 63/013,434 filed on Apr. 21, 2021 incorporated herein by reference in its entirety.

### TECHNICAL FIELD

The following relates to respirator devices, and more particularly to cartridges for elastomeric respirator devices.

### BACKGROUND

Humans have been affected by respiratory illnesses for many centuries. Common respiratory illnesses include tuberculosis, seasonal flu, pertussis, swine flu, SARS (severe acute respiratory syndrome), MERS (middle east respiratory syndrome) and COVID-19. These respiratory illnesses have been reported in all parts of the world including Asia, North America, and Europe. Many respiratory illnesses spread primarily by close person-to-person contact with symptomatic individuals (e.g., persons with fever, cough, sneezing or other respiratory symptoms). The respiratory illnesses can be spread by touching the skin of other individuals or objects contaminated with infectious droplets and then touching one's own eyes, nose, or mouth. Contamination occurs when someone with the illness coughs or sneezes droplets onto themselves, other individuals, or nearby surfaces. It also is possible that the respiratory illness can be spread further through the air by very small particles, referred to as airborne transmission.

One method to slow the spread of illness is disinfection. Surfaces which commonly harbor bacteria and/or viruses may be disinfected. In disinfection, the bacteria and/or viruses are killed such that they are not able to reproduce and cause illness. Chlorination, ozone and ultraviolet (UV) light are commonly known methods of disinfection. Chemical disinfectants such as alcohol, iodine, chlorine may also be used for disinfecting. However, these methods of disinfections are largely used for disinfecting surfaces which commonly harbor bacteria and/or viruses. These methods are generally not suitable for disinfecting humans which carry the contagion. It may also be challenging to disinfect the air used for breathing.

There are 3 main types of UV light: UVA (long wave, low energy, 315-400 nm wavelength); UVB (medium wave, medium energy, 280-315 nm wavelength); and UVC (short wave, high energy, 100-280 nm wavelength). UV-C light has been used in the past to disinfect objects. The UV-C light will effectively kill viruses, bacteria and other airborne contagion. In particular, the wavelength of 264 nm is effective at killing viruses and bacteria.

Another method to stop or slow the spread of the illnesses is through the use of respirators or face masks. Respirators provide a physical barrier which prohibits the bacteria and viruses from entering into a host's body. Since the bacteria or viruses are prohibited from entering a host's body, they are unable to reproduce and cause disease or illness. The most critical places where bacteria and viruses enter the body include: the nose, eyes, ears and mouth. Therefore, it is critical to provide a physical barrier to cover these orifices. Respirators may have half-face forms that cover only the bottom half of the face including the nose and mouth, and full-face forms that cover the entire face.

Respirators typically have a facepiece held to the wearer's head with straps, a cloth harness, or some other method. Facepieces come in many different styles and sizes, to accommodate all types of face shapes. The differences in respirator design impact the respirator assigned protection factors, i.e. the resulting degree of protection.

Particulate filtering facepiece respirators are a subset of respirators which are discarded when they become unsuitable for further use due to considerations of hygiene, excessive resistance, or physical damage. An example of a particulate filtering facepiece is shown in FIG. 1A. Powered air-purifying respirators are another subset of respirators which have a battery-powered blower that moves the airflow through filters. An example of a powered air-purifying respirator is shown in FIG. 1B.

Elastomeric respirators are yet another subset of respirators which are reusable. Elastomeric respirators comprise a facepiece as well as at least one filter cartridge. An example of an elastomeric respirator is shown in FIG. 2. The facepiece can be cleaned and reused, but the filter cartridges must be discarded and replaced when they become unsuitable for further use. Filter cartridges generally comprise layers of various chemical filters such as activated carbon layer, charcoal layer, aerosol filter layer, absorbent layer, etc. An example of the replaceable filter cartridge is shown in FIG. 3.

Respirators in this family are rated as N, R, or P for protection against oils. This rating is important in industry because some industrial oils can drastically alter or degrade the filter performance. Respirators are rated “N,” if they are Not resistant to oil, “R” if somewhat Resistant to oil, and “P” if strongly resistant to oil (oil-Proof). Furthermore, each of the N, R and P ratings have 3 varieties of filtration limits. Respirators can additionally be rather either 95, 99 or 100. Respirators rated 95 filters at least 95% of airborne particles; respirators rated 99 filters at least 99% of airborne particles; and respirators rated 100 filter at least 99.97% of airborne particles. Thus, there are nine types of disposable particulate respirators: N-95, N-99, and N-100; R-95, R-99, and R-100; and P-95, P-99, and P-100.

National Institute for Occupational Safety and Health (“NIOSH”) uses very high standards to test and approve respirators for occupational uses. NIOSH-approved disposable respirators are marked with the manufacturer's name, the part number (P/N), the filter rating (e.g., N-95), and “NIOSH.” This information is printed on the facepiece, exhalation valve cover, or head straps. While masks like N95 and others are effective, they are mostly one time use and are expensive to maintain. Providing respirators that are reusable, effective, and can be used by the general public and the healthcare industry is a challenge.

Another problem with respirators is that they do not kill the viruses and/or bacteria which cause illness. They simply provide a physical barrier which keeps the viruses and bacteria out of a host's body. However, it is still possible for some of the viruses and bacteria to make it past the physical layer and into the host's body. Therefore, there is a need for a respirator which exterminates as well as provides a physical barrier against airborne contagions.

### SUMMARY

In one aspect, a reusable respiration device is provided which is configured to kill infectious virus or other air borne contagion (examples of such viruses and bacteria include SARS, MERS, MRSA, Ebola, norovirus and C-DIFF) before it enters the user's airways. The respiration device comprises a wearable mask portion, an evacuation filter in fluid connection to the wearable mask portion; and at least one UV (ultraviolet) cartridge in fluid connection to the wearable mask portion. The UV cartridge comprises a housing having an inlet at a first end and an outlet at a second end opposite the first end; a series of UV bulbs located within the housing; and a powering means. Ambient air enters the respirator device through the inlet and is subject to UV radiation emitted from the series of UV bulbs such that the ambient air becomes sterilized air. Sterilized air exits the UV cartridge and enters the wearable mask portion via the outlet. A user is able to inhale the sterilized air entering the mask portion and exhale the used air through the evacuation filter.

### BRIEF DESCRIPTION OF THE DRAWINGS

Embodiments will now be described with reference to the appended drawings wherein:

FIG. 1A is a perspective view of a particulate filtering facepiece respirator which forms part of the prior art.

FIG. 1B is a perspective view of a powered air-purifying respirator which forms part of the prior art.

FIG. 2 is a front view of an elastomeric respirator which forms part of the prior art.

FIG. 3 is a perspective view of replaceable filter cartridges to be used with elastomeric respirators which are prior art.

FIG. 4 is a schematic cross-sectional view of the UV cartridge.

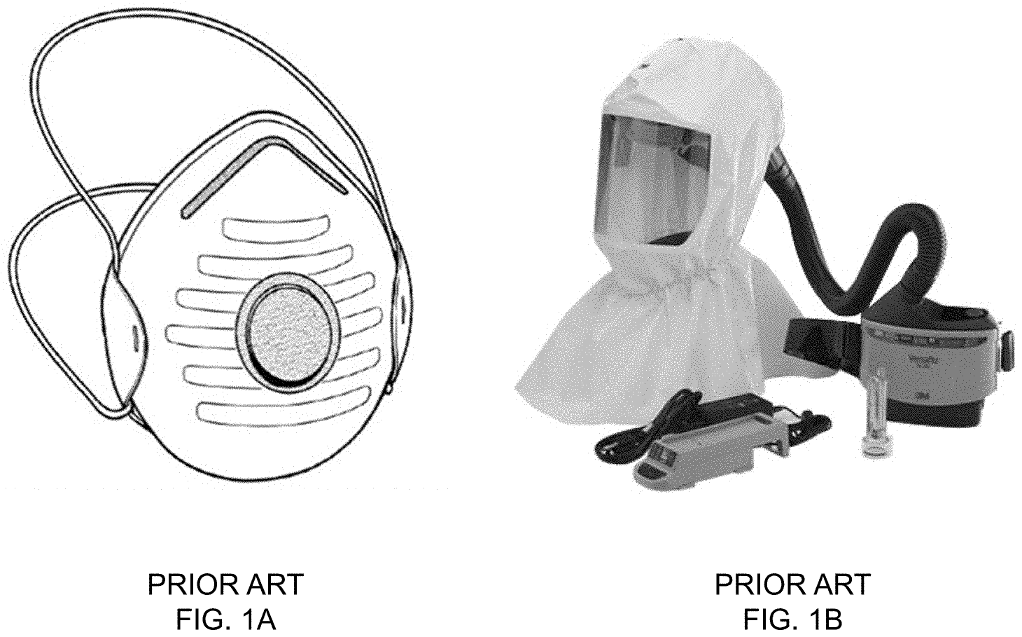

### DETAILED DESCRIPTION

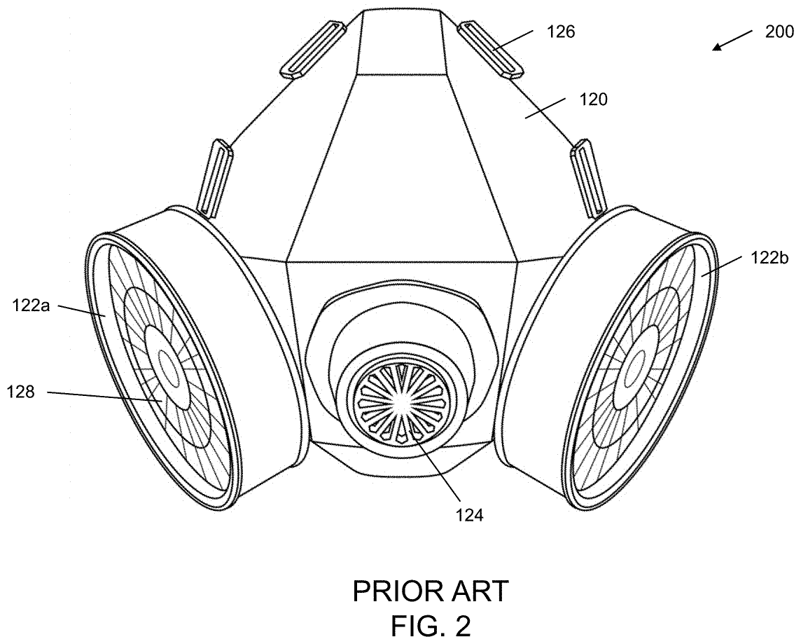

Elastomeric respirators 200, as known in the prior art, typically comprise a mask portion 120 as well as at least one filter cartridge 122, as shown in FIG. 2. The elastomeric respirator 200 may also comprise strapping means 126 from which straps can be connected to such that the respirator 200 and mask 120 can be properly fitted on a user. The mask portion 120 on an elastomeric respirator 200 can be cleaned and reused, but typically, the filter cartridges 122 have to be discarded and replaced when they become unsuitable for further use. Typical filter cartridges 122 for respirators comprise layers of various chemical filters such as an activated carbon layer, a charcoal layer, an aerosol filter layer or an absorbent layer. Ambient air enters the elastomeric respirator 200 through inlets 128 located on the filter cartridges 122 and is subject to layers of various chemical filters located within the filter cartridge 122 to produce sterilized air. Sterilized air exits the filter cartridge 122 and enters the wearable mask portion 120 where a user is able to inhale the sterilized air entering the mask portion 120. Once the user breathes the sterilized air, they are able to exhale the used air through the evacuation filter 124.

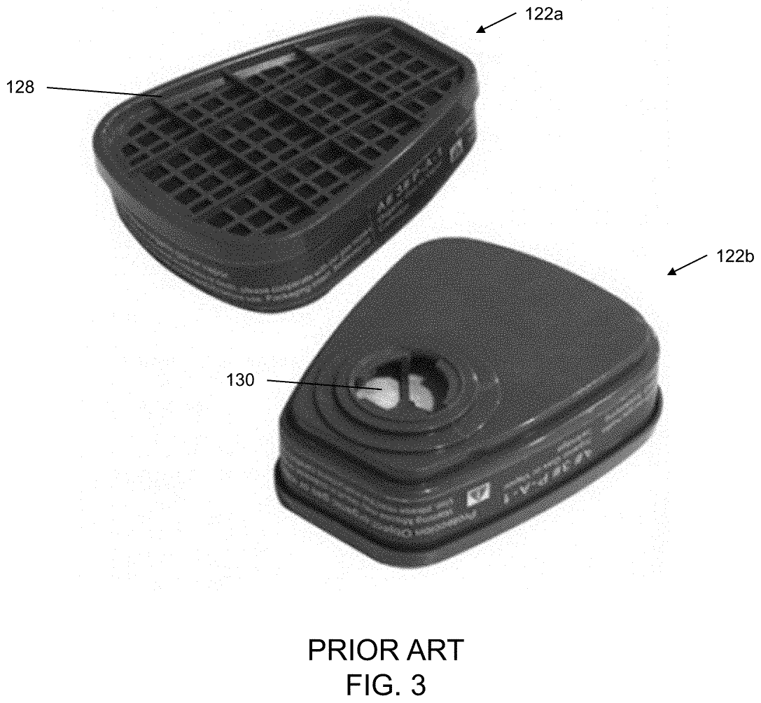

FIG. 3 provides a perspective view of replaceable filter cartridges 122 to be used with elastomeric respirators 200 which constitute prior art. The filter cartridges 122 comprise a first surface on which a plurality of inlets 128 are located. The inlets face the ambient air and allow the ambient air to enter the filter cartridge 122. On an opposite surface, the filter cartridge 122 comprises a coupling means 130 to couple to the mask portion 120. The filter cartridge 122 removably couples to the mask portion 120 such that the filter cartridges 122 can be easily removed and replaced once it is unsuitable for further use.

A UV cartridge 100 is herein provided which is capable of exterminating air-borne viruses and bacteria before they enter a user's airways. It can be appreciated that the UV cartridge 100 can replace a filter cartridge 122 of a respiration device such as an elastomeric respirator device 200. The UV cartridge 100 also comprises a couplings means 130 to couple to the mask portion of an existing elastomeric respirator 200. The UV cartridge 100 is reusable yet completely sanitary and effective whilst being easy to operate and maintain.

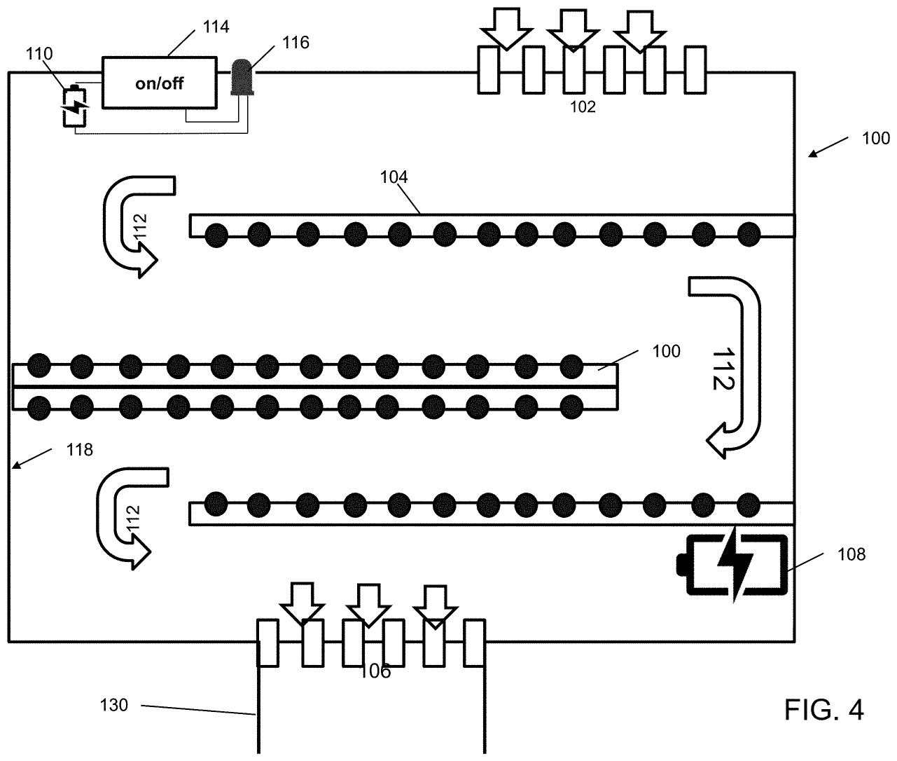

A schematic view of the UV cartridge 100 is provided in FIG. 4. The UV cartridge 100 can be used with an existing respiration device such as an elastomeric respirator device 200. The UV cartridge 100 taught herein comprises a series of UV light bulbs 104 which can be used to exterminate any air borne contagion such as viruses and/or bacteria. The UV cartridge 100 comprises at least one UV lightbulb 104, preferably a series of UV light bulbs 104. In one embodiment, the UV lightbulbs 104 are of the LED type, however any suitable UV light may be used. LED light bulbs are preferable because they are lightweight and have reported higher operating efficiencies. The LED UV lightbulbs are preferably battery-operated; however, they may be powered by any suitable method including photovoltaic, capacitive, or kinetic methods of charging. The LED UV lightbulbs are preferably light-weight such that a user may wear the respiration device without discomfort. Additionally, the UV cartridge 100 comprises UV-C light, preferably having a wavelength of 264 nm. This wavelength of light is the most effective at breaking down bacterial cell walls and viruses.

Ambient air is absorbed from an inlet 102. The ambient air is then subject to the series of LED UV bulbs 104. In this embodiment, the ambient air is subject to multiple layers of UV bulbs 104. The UV bulbs 104 are charged through a first battery 108. The first battery 108 may be rechargeable or replaceable. The longer exposure to ultraviolet light will cause a higher number of the airborne contagions to be exterminated. At an opposite end, the cleaned air exits the UV cartridge 100 and enters the user's nose, mouth and lungs through the passage 106. The flow rate of the air 112 passing through the UV cartridge 100 is primarily dependent on the user's breath. For instance, the air flow through the mask for a user taking slow breaths may be slower than the air flow through the mask for a user taking faster breaths. The user's breath will therefore ensure that the incoming ambient air passes through the various layers of UV bulbs 104 before exiting through the passage 106. Once the user breathes the air, it may be exhaled through the UV cartridge 100 or through an evacuation filter 124. It can be appreciated that the UV cartridge 100 should contain at least the volume of space that would allow ample air to enter the space and exit. For instance, the tidal volume (volume of air breathed in or out per breath) of an average user is approximately 0.5 litres.

The UV cartridge 100 may also comprise a second battery 110 and may have a coloured LED light 116 to show the status of the batteries 108, 110. For instance, the coloured LED light may show green for high charge, red for low charge, etc. Furthermore, the respiration device may also comprise an on/off switch 114 to conserve battery life of the device. The on/off switch 114 may also be a depressible button or any other suitable switch known to a person skilled in the art. It can be appreciated that the on/off switch 114 should not accidentally turn off during use and therefore should be placed accordingly. For instance, the on/off switch 114 may be placed on the side of the UV cartridge 100 where the user is not likely to accidentally engage the on/off switch 114. Other placements of the on/off switch may be possible as well. The on/off switch 114 may also include a protective member to decrease the likelihood of it being accidentally engaged. The on/off switch 114 may also be waterproof.

Additionally, the UV cartridge may be used to replace a filter cartridge of an existing elastomeric respirator. In this embodiment, the UV cartridge can be sized according to a filter cartridge such that it can easily replace the filter cartridge. For construction masks, the UV cartridge may also be equipped with a dust filter to prevent dust and particulates from entering the UVC cartridge. The dust filter may be in the form of an adapter at the outside of the UV cartridge 100.

It can be appreciated that the user should not be subject to the emitted UVC radiation from the UVC lightbulbs. As such, the UV cartridge 100 can be in fluid connection to the mask, however light from the UV bulbs will not reach the user. The internal walls 118 of the UV cartridge 100 may also include an anti-reflective (AR) coating in order to maximize photon emission from the LED UVC light bulbs. This can help increase the efficiency of the respiration device as the emitted photons of light will be reflected within the UV cartridge 100, increasing their contact with the airborne contagion and killing them faster. The UV cartridge 100 can be constructed from sapphire or fused silica. The anti-reflective coating may be applied to one or both faces of the UV cartridge 100.

Some materials including some plastics are susceptible to breakdown by UV light. Therefore, a UV stable material is preferable for the construction of the UV cartridge 100. Certain plastics such as acrylic, Ultem®, PVDF, and PTFE are inherently UV stable. Therefore, in a preferred embodiment the UV cartridge 100 is constructed from a UV-stable plastic such as acrylic, Ultem®, PVDF, or PTFE.

The mask portion of the respiration device may be half-face mask, without eye protection; or a full-face mask including eye protection. The respiration device may be particularly useful for people in health care, for example, health care workers, front line health care workers, doctors, nurses, and patients. However, it can be appreciated that other populations, such as the general public could also use the mask.

In one embodiment, a full face mask can be constructed which comprises a panel of UVC bulbs. The UVC bulbs face away from the user such that the emitted radiation cannot be felt by the user.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

Although the above principles have been described with reference to certain specific examples, various modifications thereof will be apparent to those skilled in the art as outlined in the appended claims.

1. A respirator device comprising:
a wearable mask portion;
an evacuation filter in fluid connection to the wearable mask portion; and
at least one UV (ultraviolet) cartridge in fluid connection to the wearable mask portion;
the UV cartridge comprising:
a housing having an inlet at a first end and an outlet at a second end opposite the first end;
a series of UV bulbs located within the housing; and
a means for powering said UV bulbs;
wherein ambient air enters the respirator device through the inlet and is subject to UV radiation emitted from the series of UV bulbs such that the ambient air becomes sterilized air;
the sterilized air exits the UV cartridge and enters the wearable mask portion via the outlet; and
wherein a user is able to inhale the sterilized air entering the mask portion and exhale the sterilized air through the evacuation filter.

2. The respirator device according to claim 1, wherein the UV bulbs emit UVC radiation.

3. The respirator device according to claim 2, wherein the UVC radiation has a wavelength of 264 nanometers.

4. The respirator device according to claim 1, wherein the UV cartridge is composed of an anti-reflective material.

5. The respirator device according to claim 1, wherein the UV bulbs are LED (light emitting diodes) UV bulbs.

6. The respirator device according to claim 1, wherein the means for powering said UV bulbs is a battery.

7. The respirator device according to claim 6, wherein a means for determining battery status is provided.

8. The respirator device according to claim 1, wherein the respirator device further comprises an on/off switch to engage the respirator device to have an on-status or an off-status such that the on-status permits ambient air to enter the UV cartridge and the off-status prohibits ambient air from entering the UV cartridge.

9. The respirator device according to claim 1, wherein the wearable mask portion is full face.

10. The respirator device according to claim 1, wherein a dust filter is connected to the UV cartridge to prevent dust particles from entering the UV cartridge.

11. A UV cartridge for a respirator device comprising:
a housing having an inlet at a first end and an outlet at a second end opposite the first end;
a series of UV bulbs located within the housing; and
a means for powering said UV bulbs;
wherein ambient air enters the UV cartridge through the inlet and is subject to UV radiation emitted from the series of UV bulbs such that the ambient air becomes sterilized air; and
the sterilized air exits the UV cartridge via the outlet.

12. The UV cartridge according to claim 11, wherein the UV bulbs emit UVC radiation.

13. The UV cartridge according to claim 12, wherein the UVC radiation has a wavelength of 264 nanometers.

14. The UV cartridge according to claim 11, wherein the UV cartridge is composed of an anti-reflective material.

15. The UV cartridge according to claim 11, wherein the UV bulbs are LED (light emitting diodes) UV bulbs.

16. The UV cartridge according to claim 11, wherein the means for powering said UV bulbs is a battery.

17. The UV cartridge according to claim 16, wherein a means for determining battery status is provided.

18. The UV cartridge according to claim 11, wherein the respirator device further comprises an on/off switch to engage the respirator device to have an on-status or an off-status such that the on-status permits ambient air to enter the UV cartridge and the off-status prohibits ambient air from entering the UV cartridge.

19. The respirator device according to claim 11, wherein a dust filter is connected to the UV cartridge to prevent dust particles from entering the UV cartridge.
